(19) United States
(12) Patent Application Publication
(10) Pub. No.: US 2018/0147328 A1
(43) Pub. Date: May 31, 2018

(54) Endoscopic Surgical Devices and Other Surgical Devices and Methods of Making, Especially Using Polyarylamides, Polyetherimides, Polyether Ether Ketones, and Liquid Crystal Polymers

(71) Applicant: MicroAire Surgical Instruments, LLC., Charlottesville, VA (US)

(72) Inventors: Douglas Keller, Charlottesville, VA (US); Shannon Vaughn, Charlottesville, VA (US)

(21) Appl. No.: 15/874,442

(22) Filed: Jan. 18, 2018

Related U.S. Application Data
(63) Continuation-in-part of application No. 14/843,417, filed on Sep. 2, 2015.

(51) Int. Cl. A61L 31/06; A61B 17/32

(52) U.S. Cl. CPC A61L 31/06; A61B 2017/00526; A61B 2017/320008; A61B 17/320036

(57) ABSTRACT

An endoscopic surgical device having a windowed blade case featuring an interior surface that is black or dark plastic, provides improved feasibility and ease of use.

### FIELD OF THE INVENTION

The present invention generally relates to surgical instruments, especially endoscopic surgical instruments, and is particularly related to surgical tools and procedures which can be used for the release of the transverse carpal ligament, as well as in other applications.

### BACKGROUND OF THE INVENTION

Examples of surgical tools which are useful for inspecting and manipulating tissues (e.g., cutting of the transverse carpal ligament) in a body cavity are described in U.S. Pat. No. 4,962,770 to Agee, U.S. Pat. No. 4,963,147 to Agee, U.S. Pat. No. 5,089,000 to Agee, U.S. Pat. No. 5,306,284 to Agee, and U.S. Pat. No. 7,918,784 to Wellborn et al. (Microaire Surgical Instruments, Inc.). Endoscopic instruments are used in well-established surgical procedures, such as for the release of the transverse carpal ligament. The devices include a cutting assembly for the dissection of the ligament and an endoscope with a camera system for visualization.

However, the endoscopic instrumentation currently in commercial usage is not completely free of any disadvantage or shortcoming and seemingly certain disadvantageous aspects are inherent to usage of endoscopic instrumentation. For example, the reason that patients undergo carpal and cubital tunnel releases is because the tissue in these regions is compressing the median and ulnar nerves, causing pain and numbness and loss of function. An endoscopic surgical device is used to cut ligaments and tissues to relieve that pressure. But, by inserting the device to perform the surgery, the device increases the pressure on the nerve, even for just the 5 minutes needed to perform the procedure, which can bring about potential additional pain and other temporary complications.

Another aspect of endoscopic instrumentation currently in widespread use is that the visualization aspect does have limitations that would seem to be unavoidable or not readily addressed practically. For example, when working with light from an endoscope and the endoscope's lens within a body cavity, moisture and light reflection on metal surfaces seemingly inevitably will complicate visibility.

In De Faria-Correa, “Endoscope viewing cannula and surgical techniques,” U.S. Pat. No. 5,448,990 issued Sep. 12, 1995 (Very Inventive Physicians, Inc.), there was suggestion to coat the interior of a housing with black paint to eliminate back-scattering degradation of an image when an endoscope is used. Painting interiors of housings is not a current practice in the industry. Paint or ink is susceptible of separating from the device and being left inside a patient. Also temperature and humidity can affect bonding of paint to a surface and painted surfaces tend to have less good shelf lives than unpainted synthetic solid surfaces. Painted surfaces further can be undesirable if a device, after having been used in a surgery, is going to be irradiated and reused in a future surgery.

Some in the industry have taken a direction of trying to reduce unwanted light reflection and glare off of the metal surfaces of the instrumentation by rough-blasting the metal surfaces so that they are less like mirrors. But roughened surfaces in medical instrumentation that is to be sterilized for reuse may not be well-received, in that a non-smooth surface may be considered more uncertain to fully clean compared to a smooth surface.

Another aspect of visualization with existing endoscopic instruments is that movement (especially rotational movement) of the blade case within the patient has been needed to orient the endoscope's lens to achieve the desired image. While a user might, in the abstract, theoretically want to be able to achieve a desired visual image without moving the blade case inside the patient, with the current instrumentation, without rotation and movement of the blade case within the patient, no useable visual view is captured.

Also, a blade case of current endoscopic instrumentation occupies a certain space within the patient and when that space is needed for another surgical tool that needs that space to perform its respective function, the blade case must be removed and reinserted.

### SUMMARY OF THE INVENTION

It is an object of the invention to address the above-mentioned disadvantages and shortcomings of existing endoscopic surgical tools.

It further is an object of the invention to provide an endoscopic surgical tool relatively unsusceptible to difficulties with glare and reflected light.

It is another object of the invention to provide an endoscopic surgical tool with minimized needs to be rotated inside a patient to achieve visualization.

Also it is an object of the invention to provide an endoscopic surgical tool that can remain inside a patient in contexts where previous endoscopic surgical tools would need to be removed to make way for another surgical instrument and then reinserted when the space was again made available by removal of that other instrument.

The invention in a preferred embodiment provides a surgical device, comprising a blade case, wherein an interior surface of the blade case is light-absorbing (such as, e.g., an interior surface of a polyarylamide blade case; an interior surface of a polyetherimide blade case; an interior surface of a Polyether ether ketone (PEEK) blade case; an interior surface of a liquid crystal polymer blade case, an interior surface of a plastic case, wherein the plastic is in a form of a polymeric structure, and the polymeric structure has incorporated therein pigment particles, the pigment particles being distributed throughout the polymeric structure; etc.), such as, e.g., inventive surgical devices in which the interior surface of the blade case has a black color (such as, e.g., a black color of a polyarylamide; a black color of a polyetherimide; a black color of a Polyether ether ketone (PEEK); a black color of a liquid crystal polymer; etc.); inventive surgical devices wherein the interior surface of the blade case has a dark color (such as, e.g., a dark color of a polyarylamide; a dark color of a polyetherimide; a dark color of a Polyether ether ketone (PEEK); a dark color of a liquid crystal polymer; etc.); inventive surgical devices wherein the blade case comprises a black plastic solid shape (such as, e.g., a black shaped polyarylamide; a black shaped polyetherimide; a black shaped a Polyether ether ketone (PEEK); a black shaped liquid crystal polymer; etc.); inventive surgical devices wherein the interior surface comprises a black plastic (such as, e.g., a black polyarylamide; a black polyetherimide; a black a Polyether ether ketone (PEEK); a black liquid crystal polymer; etc.); inventive surgical devices wherein the light-absorbing interior surface contains no painted layer and no paint and no ink; inventive surgical devices comprising a blade case without an acrylic contained therein; and other inventive surgical devices.

In another preferred embodiment, the invention provides a surgical device comprising an interior surface on which light will be shined during surgery, wherein the interior surface belongs to a component selected from the group consisting of a cannula and a blade case; and wherein the interior surface is light-absorbing (such as, e.g., an interior surface that comprises a light-absorbing material).

The invention in another preferred embodiment provides a surgical device (such as, e.g., an endoscopic device) comprising an interior surface on which light will be shined during surgery, wherein the interior surface belongs to a component selected from the group consisting of a cannula and a blade case; and wherein the interior surface is not light-reflecting.

Referring to another preferred embodiment, the invention provides a method of constructing a surgical device (such as, e.g., an endoscopic surgical device), comprising: for a surface of the surgical device on which light will be shined during surgery, constructing the surface as a light-absorbing surface, such as, e.g., inventive methods wherein the light-absorbing surface is constructed without performing a step of rough polishing or grit-blasting metal; inventive methods wherein the light-absorbing surface is constructed without performing a painting step; inventive methods wherein the light-absorbing surface is integral with a component formed from a black material or a dark material; inventive methods wherein the constructing comprises forming a non-layered integral part from a black material or a dark material, wherein the light-absorbing surface is a top surface of the black material or dark material; inventive methods wherein the constructing comprises forming a starting material into a shape that when cooled and hardened is a black or dark plastic solid (such as, e.g., a black or dark polyarylamide; a black or dark polyetherimide; a black or dark a Polyether ether ketone (PEEK); a black or dark liquid crystal polymer; etc.); inventive methods wherein the surface comprises a surface of a blade case; etc.

The invention in another preferred embodiment provides a surgical device (such as, e.g., an endoscopic surgical device), comprising: a windowed blade case comprising at least one window, the blade case having a size that accommodates passage therein of an endoscope, such as, e.g., inventive surgical devices wherein the blade case is opaque; inventive surgical devices wherein the at least one window is positioned at a tip of the endoscope; inventive surgical devices wherein the endoscope is rotatable; inventive surgical devices wherein the at least one window comprises exactly one window; inventive surgical devices wherein the at least one window comprises two windows; inventive surgical devices wherein the at least one window comprises three windows; inventive surgical devices wherein the at least one window comprises a first window and a second window, wherein the first window and the second window differ as to one or both of size and shape; inventive surgical devices wherein the at least one window is defined by a clear solid section; inventive surgical devices comprising a first window disposed on a first side of the blade case, a second window disposed on a second side of the blade case, and a third window disposed on a bottom surface of the blade case; and other inventive surgical devices.

In another preferred invention, the invention provides a method of operating an endoscope, comprising steps of: during surgery on a patient, positioning a windowed blade case inside the patient, wherein the windowed blade case comprises at least a first window (such as, e.g., a first window that comprises open space; a first window that comprises clear plastic); through the first window, performing a certain step.

The invention in another preferred embodiment provides a surgical device (such as, e.g., an endoscopic device), comprising: a blade case comprising a clear first section (such as, e.g., a clear first section having a length dimension in a range of about 2.0-3.5 inches; a width dimension in a range of about 0.15-0.25 inches; and a thickness dimension in a range of about 0.010-0.025 inches) and a light-absorbing second section; such as, e.g., inventive surgical devices wherein the clear first section is a top section of the blade case and the light-absorbing second section is a bottom section of the blade case.

In another preferred embodiment, the invention provides a method of previewing tissue to be cut during surgery (such as, e.g., carpal tunnel release surgery; cubital tunnel release surgery; endoscopic surgery; arthroscopic surgery; minimally invasive surgery (e.g., minimally invasive surgery where no dermal incision exceeds about 3 cm; minimally invasive surgery where dermal incisions are in a range of about 1-1.5 cm; etc.), the method comprising: previewing the tissue to be cut through a window of a blade case or a cannula; such as, e.g., inventive methods wherein the previewing step is performed without inserting and removing the blade case or cannula multiple times; inventive methods wherein the blade case or cannula is opaque; inventive methods wherein the window is located at a top of the blade case or cannula; inventive methods further comprising, when previewing is performed, moving the endoscope up and down a length of the tissue to be cut; and other inventive methods.

The invention in another preferred embodiment provides a surgical device (such as, e.g., an endoscopic device) comprising: a blade case comprising at least one concavity on an exterior surface thereon, such as, e.g., inventive surgical devices wherein the at least one concavity extends lengthwise along the exterior surface of the blade case; inventive surgical devices wherein the blade case is a flanged blade case, comprising at least one external wall that is concave; inventive surgical devices comprising a set of external walls that are concave; inventive surgical devices comprising an external sidewall that is concave; inventive surgical devices comprising at least two external sidewalls that are concave; inventive surgical devices wherein exactly two concave external sidewalls are included (such as, e.g., inventive surgical devices further comprising a non-concave top wall and a non-concave bottom wall); inventive surgical devices comprising an external bottom wall that is concave (such as, e.g., inventive surgical devices wherein the non-concave top wall has a flat surface); etc.

In another preferred embodiment the invention provides a surgical device (such as, e.g., an endoscopic device) comprising a blade case ending in an edged tip, wherein the edged tip comprises a scraper that extends along a longitudinal axis of the blade case, the scraper being integrally a part of the blade case; such as, e.g., inventive surgical devices in which the scraper is defined by a shape selected from the group consisting of a flared shape; a protrusion; and a swept ridge; inventive surgical devices wherein no blade is included in the edged tip; inventive surgical devices wherein the edged tip is rounded; etc.

The invention in another preferred embodiment provides a method of using a surgical device, comprising the step of: scraping synovium during endoscopic carpal tunnel surgery, wherein the scraping is performed by the surgical device, and the surgical device is also useable for splitting muscle near fascia present during endoscopic cubital tunnel surgery; such as inventive methods further comprising splitting muscle near fascia present during endoscopic cubital tunnel surgery, wherein the muscle-splitting is performed by the same surgical device that performs the synovium-scraping; etc.

In another preferred embodiment, the invention provides a method of clearing tissue in endoscopic carpal tunnel surgery, comprising: scraping synovium away by bringing an edged tip of a blade case in contact with the synovium, while an endoscope is in place illuminating the synovium during the scraping step; and/or splitting muscle for cubital procedures by contacting the edged tip of the blade case with the muscle.

Referring to another preferred embodiment, the invention provides an endoscopic surgical device, comprising: a blade; a blade case that in an unused condition is attachable to a first handpiece; and a releasable blocking tab that moves between two positions (such as, e.g., a releasable blocking tab that is located on the blade case; a releasable blocking tab that is released by ejection of the blade case from the handpiece, wherein the released tab forms a physical block sized to prevent the blade case from being reattached to the first handpiece or attached to a second handpiece; etc.).

The invention in another preferred embodiment provides an endoscopic surgical device, comprising: a blade; and a usage indicator, wherein before the blade is used for a first time, the usage indicator occupies an internal position unseen by one viewing the surgical device, and upon the blade being used for the first time, the usage indicator moves to an external position that can be seen by one viewing the surgical device.

In another preferred embodiment, the invention provides an endoscopic surgical device, comprising a blade case, wherein the blade case is selected from the group consisting of: (1) a blade case that occupies a volume less than 4 cm³, and/or has a cross-section not more than 0.36 cm² and/or has a height not more than 0.54 cm; (2) a blade case, wherein the blade case occupies a volume less than 4 cm³; (3) a blade case, wherein the blade case has a cross-section not more than 0.36 cm²; (4) a blade case, wherein the blade case has a height not more than 0.54 cm, (5) a blade case, wherein the blade case occupies a maximum external volume per unit length less than 0.055 in², with a height to width ratio less than 80%, such as, e.g., inventive endoscopic surgical devices in which the blade case occupies a volume of not more than 3.41 cm³; inventive endoscopic surgical devices in which the blade case has a height not more than 0.54 cm; inventive surgical devices comprising a flange; inventive surgical devices comprising a blade case having a length of at least about 9 cm; etc.

### BRIEF DESCRIPTION OF THE DRAWINGS

The invention may be appreciated by reference to the figures, which are not necessarily drawn to scale:

FIGS. 1-1A are perspective views of an exemplary surgical device 1 according to the invention, comprising blade 100 in, respectively, retracted position and raised position.

FIGS. 2-2A are cross-sectional views of blade case 2 according to the invention useable in surgical device 1 of FIG. 1, depicting a hollow interior circular section defined by interior surface 3 of blade case 2.

FIG. 3 is a lengthwise cross-sectional view of a blade case 12 according to the invention, useable in surgical device 1 of FIG. 1. Blade case 12 has length 12L.

FIG. 3A is lengthwise cross-sectional view of blade case 12 of FIG. 3 rotated 90 degrees about the lengthwise 12L axis, showing window 11.

FIG. 3B is a close-up perspective view of windows 7, 9, 11 (FIGS. 3-3A) in blade case 12 as a top view. Light cone 10 emanates from the endoscope in a straight up, standard position.

FIG. 3C is a close-up perspective view corresponding to FIG. 3B, as a side view. Light cone 10′ emanates from the endoscope rotated 45 degrees to one side, to view side tissue.

FIG. 3D is a close-up perspective view corresponding to FIGS. 3B-3C, as a bottom view. Light cone 10″ emanates from the endoscope rotated 180 degrees to view tissue beneath the blade case 12.

FIG. 4 is a cross-sectional view of a clear-topped blade case 22 according to the invention, useable in surgical device 1 (FIG. 1) and having blade case length 22L.

FIG. 4A is a width-wise cross-sectional view of the blade case 22 of FIG. 4.

FIG. 5 is a lengthwise cross-sectional view of a clear-bottomed blade case 23 according to the invention, useable in surgical device 1 (FIG. 1) and having blade case length 23L.

FIG. 5A is a width-wise cross-sectional view of the blade case 23 of FIG. 5.

FIG. 6 is a lengthwise cross-sectional view of a flanged blade case 32 useable in surgical device 1 (FIG. 1) and having blade case length 32L.

FIG. 6A is an enlarged width-wise cross-sectional view of flanged blade case 32 (FIG. 6).

FIG. 6B corresponds to FIG. 6A, and depicts concavity depth 16D.

FIG. 7 is a cross-sectional view of ridges 21 formed into a concavity on an exterior surface of a blade case in an embodiment of the invention.

FIG. 8 is a cross-sectional view of a hooked edge 24 formed into a concavity on an exterior surface of a blade case in an embodiment of the invention.

FIG. 9 is a top view of a scraper-tipped blade case 42 useable in surgical device 1 (FIG. 1) and having blade case length 42L.

FIG. 10 is a side view of blade case 42 (FIG. 9) comprising scraper tip 40 according to the invention.

FIG. 10A is an enlarged view including scraper tip 40 from FIG. 10.

FIG. 11 is an exploded perspective view in an inventive embodiment of parts comprising main blade case body 50, windows 7, 9, 11 (FIGS. 3-3A), a clear top 4 of the blade case, a clear bottom 4A of the blade case, a concave exterior surface 16 of the blade case, and a scraper tip 40.

FIG. 11A is an assembled perspective view corresponding to FIG. 11, depicting a surgical device according to an embodiment of the invention.

FIG. 12 is a diagrammatic representation of a cross-sectional view of pigment dispersion in a polymer matrix in an embodiment of the invention, with O representing polymer matrix and ● representing pigment particle.

### DETAILED DESCRIPTION OF A PREFERRED EMBODIMENT

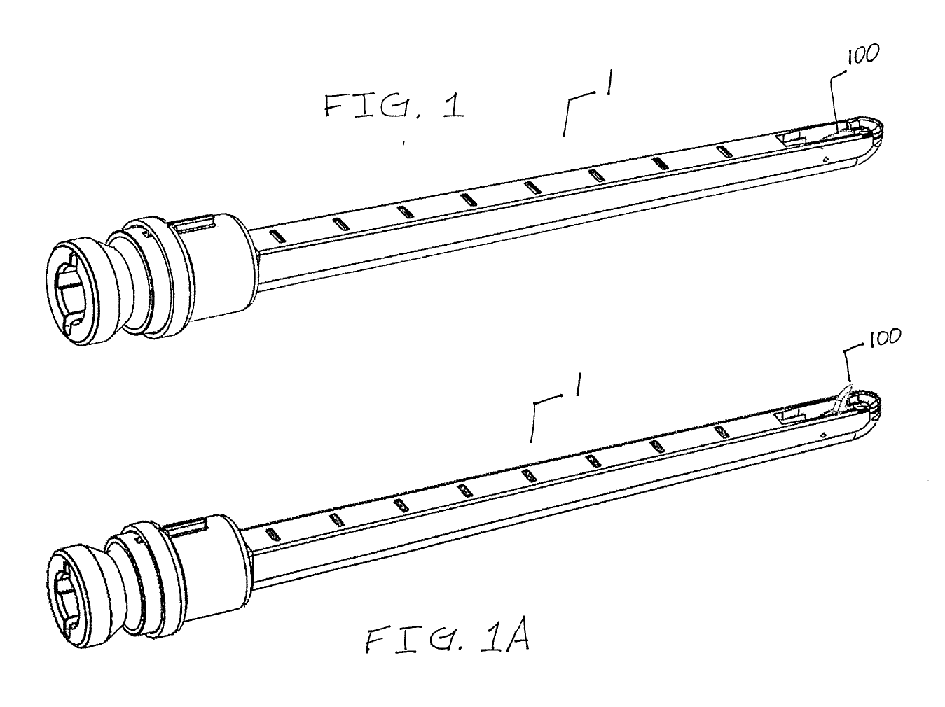

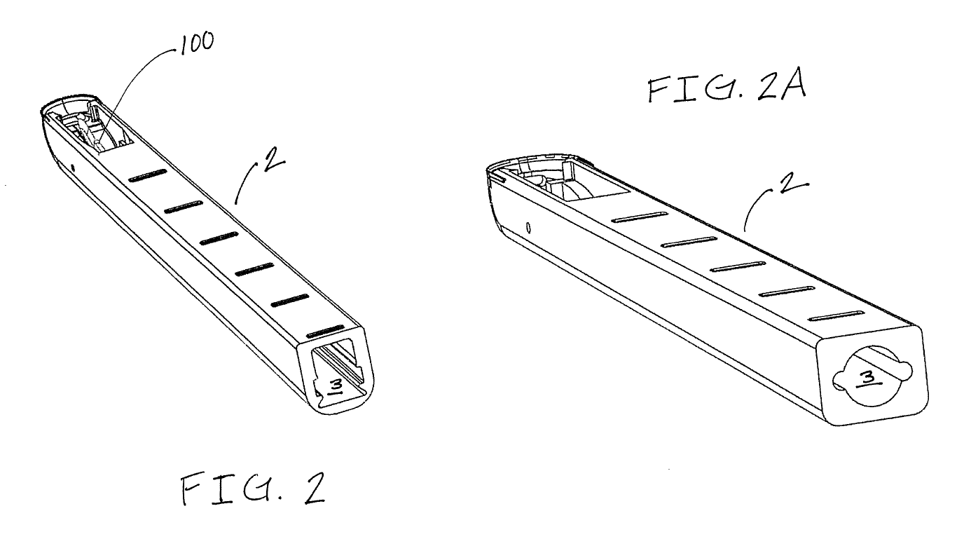

The invention provides for certain advances and improvements in surgical tools, surgical devices, and methods of using such surgical tools and devices. In the invention, a preferred example of a surgical device is a surgical device (such as surgical device 1 in FIG. 1) comprising an endoscopic device, also referred to as “an endoscopic surgical device”. The invention particularly improves upon surgical devices comprising a blade case (such as blade case 2 in FIG. 2), such as endoscopic devices comprising a blade case. Using combinations of the innovations provided herein is preferred but not mandatory.

For the interior surface 3 of the blade case 2, a light-absorbing interior surface is particularly preferred. Examples of a “light absorbing” surface are, e.g., a surface having low reflectance; a surface having low reflectivity; a surface having low albedo; a non-reflective surface; a surface having a measured light reflectance value ≤20% when visible light is shined thereon; etc. By a measured light reflectance value when visible light is shined thereon, we are referring to the light reflectance that is measured upon shining visible light in a range of 400-700 nm by an endoscopic instrument set to full intensity. The most preferred examples of a light absorbing surface for use in the invention are a black surface or a dark surface.

To construct the light absorbing surface, painting or any process resembling painting (such as silk-screening or inking or pad printing) is not preferred and should be avoided.

For constructing the light absorbing surface, a black or dark plastic is preferred, such as a black or dark polyarylamide; a black or dark polyetherimide; a black or dark a Polyether ether ketone (PEEK); a black or dark liquid crystal polymer; etc. Non-plastics that can be constructed into a black or dark solid also are useable to construct the light absorbing surface.

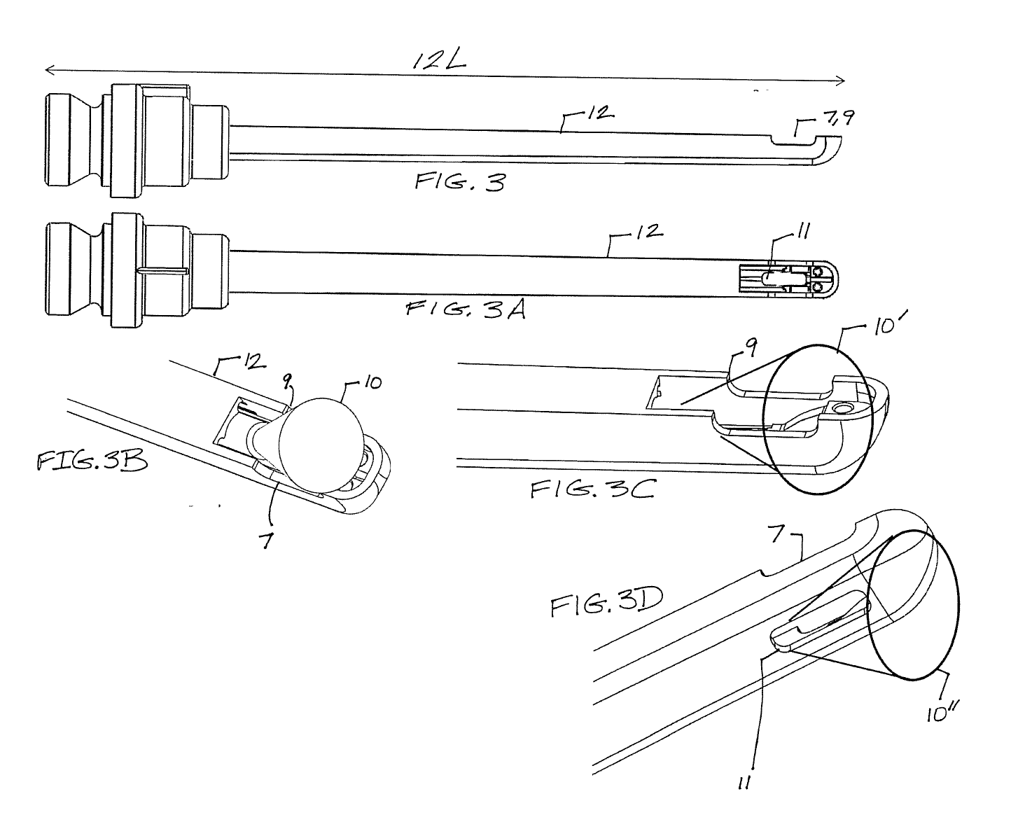

For the blade case 2, a windowed blade case (such as windowed blade case 12 in FIGS. 3-3D comprising windows 7, 9, 11) is particularly preferred, with a set of three windows being a most preferred configuration.

For the set of three windows 7, 9, 11, a preferred example of the first window 11's dimensions is about 0.1-0.3 inches long by 0.05-0.1 inches wide, with the second and third windows 7, 9 each respectively being about 0.2-0.5 inches long by 0.05-0.1 inches tall. In another example, the first window has a length dimension about 7 mm and a width dimension in a range of about 1.5-2 mm, and the second window and third window have a length dimension about 7 mm and a width dimension in a range of about 1.5-2 mm.

Referring to FIGS. 3A-3D, light cones 10, 10′, 10″ are depicted in a context of window 11 and windows 7, 9. Light cones 10, 10′, 10″ indicate the visual cone of the endoscope in three different positions.

Windows 7, 9, 11 are easily constructed such as by cutting holes through blade case 12 on the sides and bottom.

Through windows 7, 9, 11, soft tissue anatomy is viewable during surgery (with a rotating endoscope).

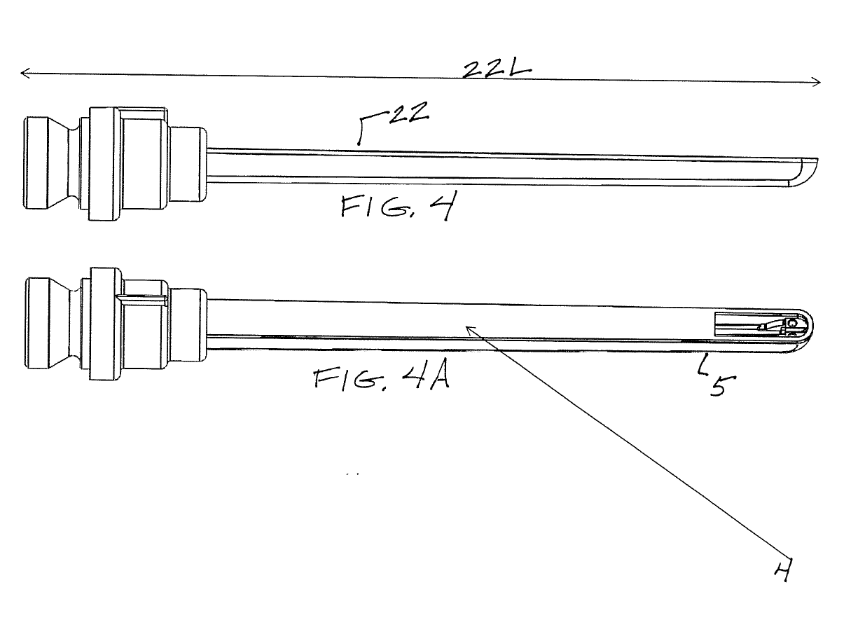

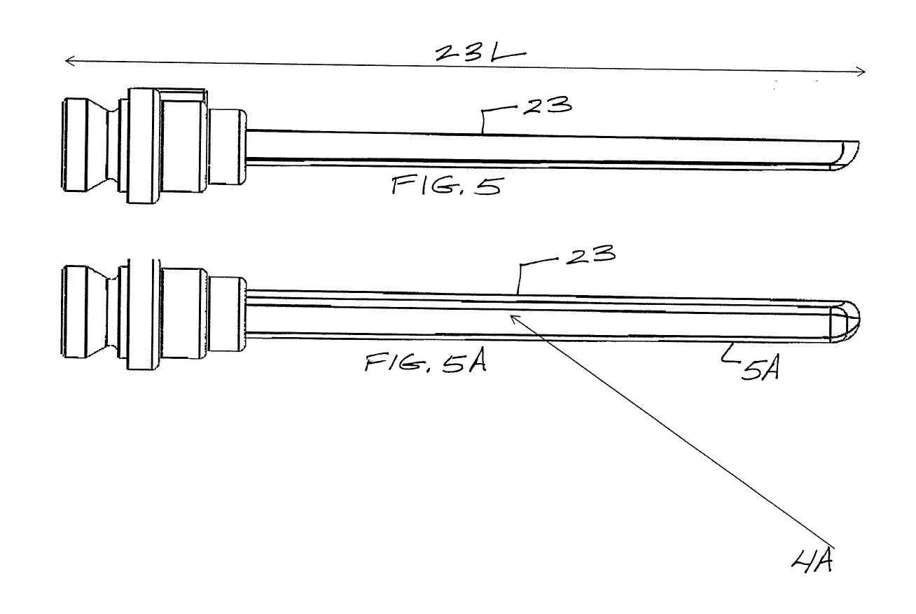

For blade case 2, a clear-topped blade case such as blade case 22 (FIGS. 4-4A) is preferred. Clear top 4 in blade case 22 allows visualization via endoscope along an entire length 22L of blade case 22.

For blade case 2, a clear-bottomed blade case such as blade case 23 (FIGS. 5-5A) is preferred. Clear bottom 4A allows visualization via endoscope along the entire length 23L of blade case 23.

Preferably blade case 2 is both clear-bottomed and clear-topped.

For constructing body 5 of blade case 22 and body 5A of blade case 23, preferably a relatively stronger opaque material is used. Top 4 and bottom 4A are constructed from clear material that allows visualization via an endoscope as the endoscope is moved along lengths 22L, 23L of blade case 22, 23. The combination of materials used for top 4 and body 5 (and bottom 4A and body 5A) is selected to maintain stiffness of the opaque design while allowing greater visualization than if top 4 (and bottom 4A) were not clear. An example of a range of thickness for clear top 4 or bottom 4A is about 0.020-0.040 inches thick.

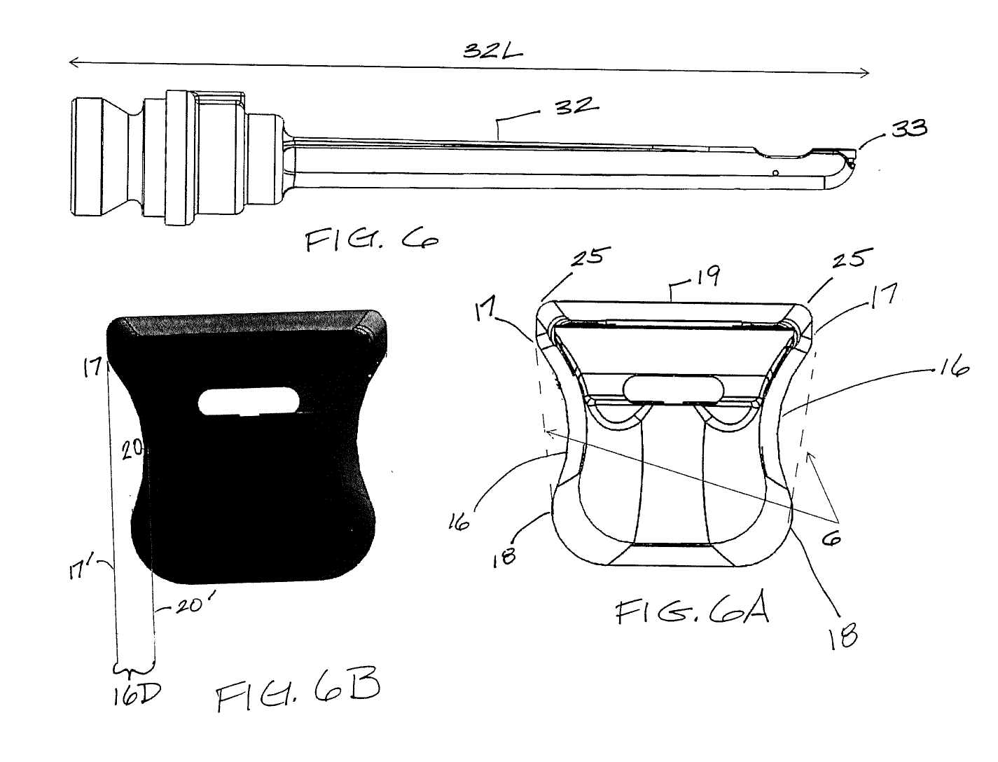

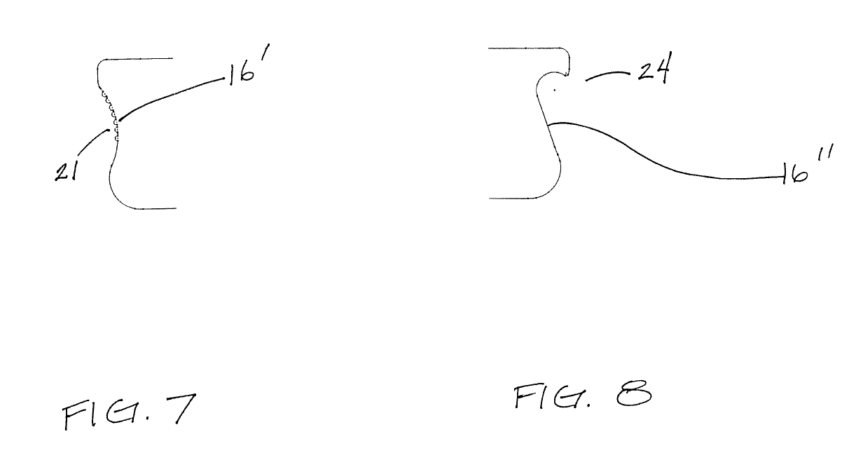

For blade case 2, a flanged blade case such as blade case 32 (FIGS. 6-6B) is preferred. In FIG. 6A, the linear profile 6 of blade cases of currently-sold endoscopic instrumentation is shown as dotted lines. The invention provides for AVOIDING the linear profile 6 and instead constructing concave surfaces 16 (FIG. 6A). By forming concave surfaces 16, the cross-sectional profile in the invention approximates an hourglass shape. Concave surfaces 16 run axially along exterior of blade case 32. A concavity is defined by a concave surface 16 between non-concave surface sections 17, 18. The concavities create pockets for soft tissue to rest and hurdles for the same tissue to overcome to slide over the top 19 of blade case 32.

In FIGS. 6-6B, construction of a concavity into both side surfaces of a blade case is illustrative; in other embodiments, a concavity is constructed into only one side surface of a blade case.

Concavity depth 16D (FIG. 6B) is the distance between, on the one hand, a line 17′ defined by the non-concave section 17, and, on the other hand, a line 20′ including the most concave point 20 of the concave surface 16 and drawn parallel to line 17′. For a blade case having length 32L of about 3.75-3.8 inches, a preferred range for concavity depth 16D is about 0.025-0.050 inches.

Examples of a length of a concavity are, e.g., a length equal to a full length of a blade case; a length less than a full length of a blade case. When the concavity extends less than a full length of the blade case, for the concavity length to begin at tip 33 (FIG. 6) of blade case 32 and extend backwards from the tip 33 at least about a length equal to half the blade case 32's length is preferred.

In FIGS. 6-6B, construction of a concavity into a side surface of a blade case is illustrative; in other embodiments, a concavity is constructed into a bottom surface of a blade case. For example, a concave bottom surface of a blade case is considered useful particularly for working with the ulnar nerve in cubital tunnel release surgeries.

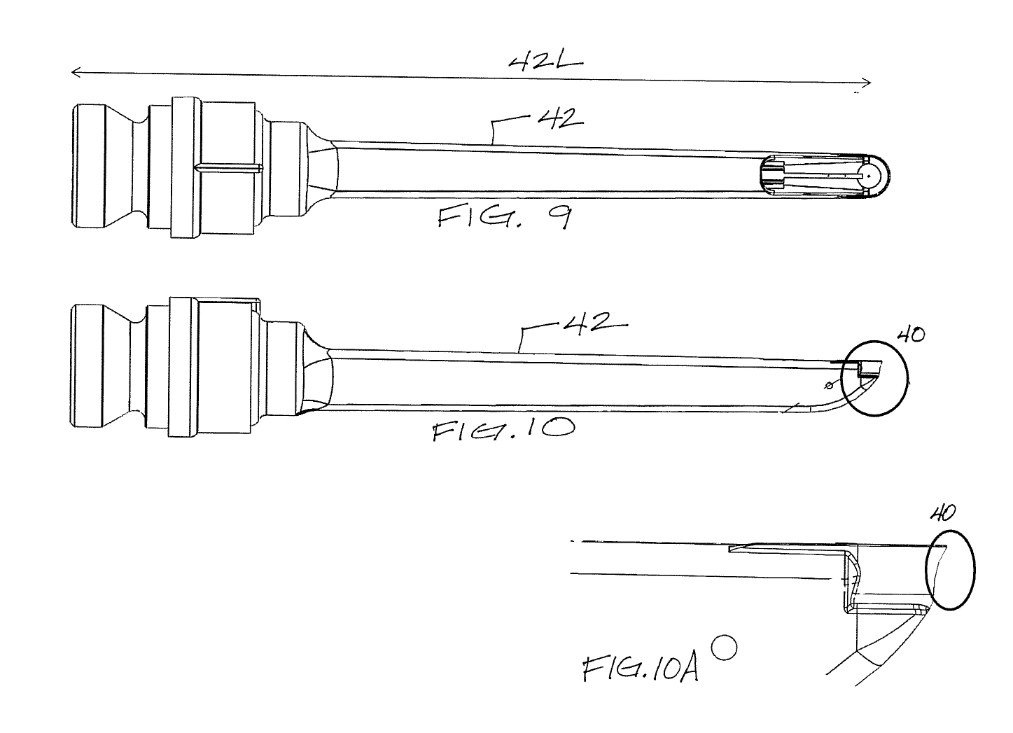

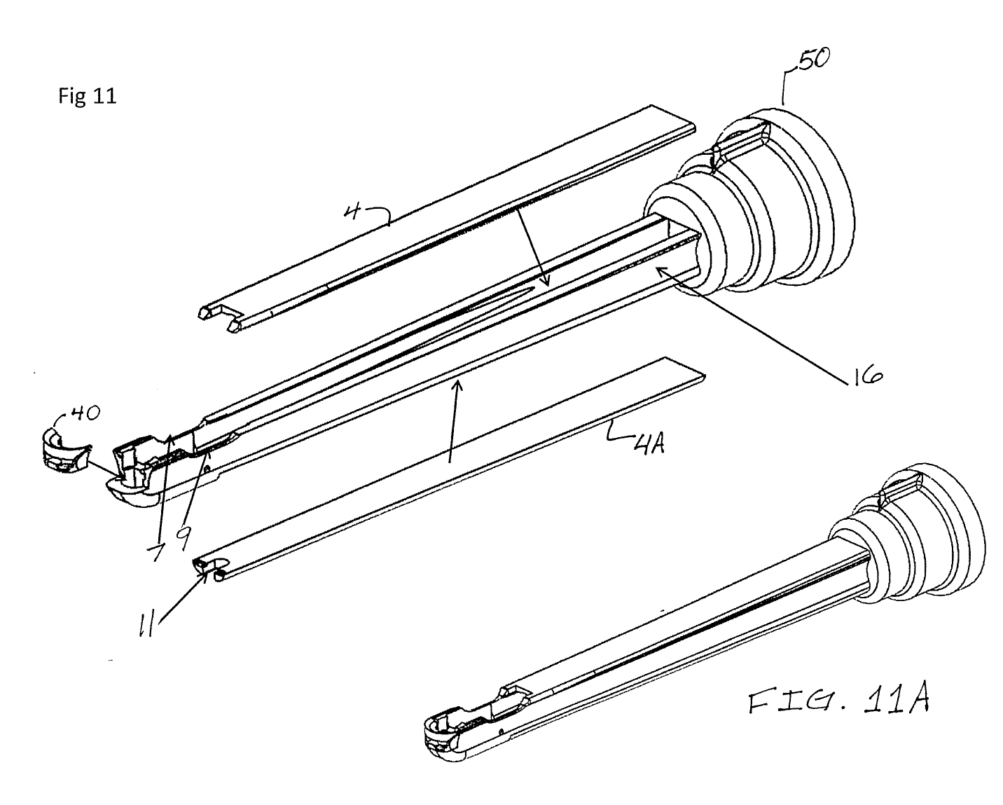

In FIG. 6A, the concave surface 16 is illustrated as smooth but it will be appreciated that the concave surface is not required to be smooth in all embodiments. For example, in some embodiments ridges 21 (FIG. 7) are formed as part of concave surface 16′. As another example, in other embodiments, surface 16″ is generally-concave without being fully symmetrical, such as a concavity defined by a hooked edge 24 (FIG. 8).

As a consequence of the concave surfaces 16, flanges 25 (FIG. 6A) are formed.

An example of a shape of a tip of the blade case is a rounded edged tip, such as, e.g., a filleted, rounded-edge shape that forms a 90-180° total arc around the distal tip of the cannula, with a fillet radius of 0.001-0.010 inch.

Preferably a scraper tip such as scraper tip 40 (FIGS. 10-10A) is included in the blade case.

Preferably scraper tip 40 is distally-flared with a sharpened edge for scraping synovium and other tissue present along the top plane of the blade case 42 during carpal tunnel surgery. As to degree of sharpness of scraper tip 40, preferably scraper tip 40 is not sharp enough to cut synovium and other biological tissue, but is sharp enough to scrape biological tissue away form the transverse ligament.

Advantageously a flared scraper tip such as scraper tip 40 also can be used to split muscle without cutting, during cubital tunnel surgery.

An endoscopic surgical instrument comprising a distally-flared scraper tip such as scraper tip 40 advantageously can remain in place within a patient when certain scraping is needed, without needing to be retracted to make room for a separate scraper to be used.

The invention may be further appreciated with reference to the following examples, without the invention being limited thereto.

### EXAMPLE 1

In this example, an inventive endoscopic surgical device according to the figures herein comprises a blade case that occupies a maximum external volume per unit length (V/L) less than 0.055 in², with a height to width (H/W) ratio less than 80%. V/L will be appreciated to essentially reflect cross-sectional area. We refer herein to “maximum” because, for injection molded plastic parts, some amount of draft is always to be expected along the sidewalls, corresponding to reduced part size moving from the hub to the tip.

| | |
|---|---|
| COMPARISON EX.* | INVENTIVE EX. |

| | | | | |
|---|---|---|---|---|
| W | 0.25 | in | 0.26 | in |
| H | 0.29 | in | 0.21 | in |
| L | 2.75 | in | at least 3.75 | in |

| H/W | 112% | | less than 80% | |
|---|---|---|---|---|
| Max. V/L | 0.075 | in² | 0.055 | in² |

*Commercially sold endoscopic surgical device

By contrast to the inventive example with H/W 80% or less (i.e., wider than tall), blade cases of endoscopic surgical devices currently sold have H/W 112% (i.e., taller than wide). Because this inventive example has H/W below 100%, the stiffness and strength of the blade case is reduced compared to the case with H/W 112%. To bring the stiffness and strength of the blade case to the requisite level, use of the further features of the figures herein is strongly preferred.

A reduction of 10% of the maximum V/L of commercially available endoscopic surgical devices would be considered a significant improvement by those in the industry. The context in which V/L is contemplated by those in the industry is as follows. The reason that patients undergo carpal and cubital tunnel releases is because the tissue in these regions is compressing the median and ulnar nerves, causing pain and numbness and loss of function. An endoscopic surgical device is used to cut ligaments and tissues to relieve that pressure. But, by inserting the device to perform the surgery, the device increases the pressure on the nerve, even for just the 5 minutes needed to perform the procedure, which can bring about potential additional pain and other temporary complications. The present invention's reduction of the device's V/L is highly advantageous, in that reduced V/L has an immediate and direct reduction in the pressure exerted by the device on the nerve during surgery.

### EXAMPLE 2

In this example is used a main blade case body 50 having integrally formed therein scraper tip 40 and comprising windows 7, 9 and concave walls 16. The main case body 50 in this example is opaque black, formed from a relatively-stronger material than used for clear sections 4, 4A.

The main case body 50, clear top section 4, clear bottom section 4A (comprising window 11) are assembled into the surgical device of FIG. 11A.

### EXAMPLE 2A

Example 2 is constructed, using polyarylamide for the opaque black main case body 50, and using acrylic for the clear sections 4, 4A.

### EXAMPLE 3

In this example, plastic resins are formulated without glass.

### EXAMPLE 3A

In this example, plastic resins are formulated with glass, up to 60% glass-filled.

### EXAMPLE 4

In this example, polymer pellets are purchased commercially, as a starting material to melt and use in a melt-and-mold process. The melted polymer is used in a mold process by which is constructed a desired plastic shape such as a blade case.

### EXAMPLE 4A

In this example, the polymer pellets as-purchased commercially have a black or a dark color (because the pellet-manufacturer introduced pigments (also called “colorants”) into the melt before cooling into a solid and then pelletizing the cooled solid). The as-purchased black, or dark, pellets are melted-down, and the melted polymer is used in a mold process to construct a desired plastic shape such as a blade case.

### EXAMPLE 4B

In this example, polymer pellets as-purchased commercially have a ‘natural’ color in a range of about between an off-white color and an amber color. The as-purchased natural-colored polymer pellets are melted, followed by adding pigments (also called “colorants”), thereby darkening the melted polymer, preferably to black. The black or dark-colored melted polymer is used in a mold process to construct a desired plastic shape such as a blade case.

### EXAMPLE 5

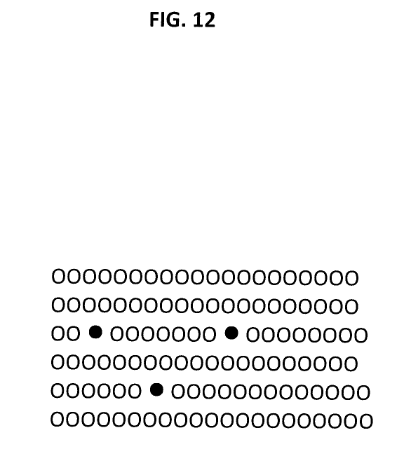

Examples of pigments useable in dispersion processes to be dispersed into a polymeric structure, to thereby darken or blacken a polymer, include organic pigments and inorganic pigments. (See FIG. 12)

### EXAMPLE 5A

A carbon black pigment

### EXAMPLE 5B

A perylene black pigment

### EXAMPLE 5C

An iron oxide black pigment.

### EXAMPLE 5D

A chrome oxide black pigment.

### EXAMPLE 5E

A manganese oxide black pigment.

### EXAMPLE 5F

A strontium iron manganese black pigment.

While the invention has been described in terms of its preferred embodiments, those skilled in the art will recognize that the invention can be practiced with modification within the spirit and scope of the appended claims.

What we claim as our invention is:

1. A surgical device, comprising:
a blade case, wherein an interior surface of the blade case is light-absorbing and comprises a plastic selected from the group consisting of:
a polyarylamide;
a polyetherimide;
a Polyether ether ketone (PEEK);
a liquid crystal polymer.

2. The surgical device of claim 1, wherein the light-absorbing interior surface contains no painted layer and no paint and no ink.

3. The surgical device of claim 1, comprising a blade case without an acrylic contained therein.

4. The surgical device of claim 1, comprising a plastic resin, wherein a constitution of a glass-filling in the plastic resin is in a range of 0 to 60% glass-filled.

5. The surgical device of claim 1, wherein the plastic is in a form of a polymeric structure, and the polymeric structure has incorporated therein pigment particles, the pigment particles being distributed throughout the polymeric structure.

6. A method of constructing a surgical device, comprising:
for a surface of the surgical device on which light will be shined during surgery, constructing the surface as a light-absorbing surface, wherein the light-absorbing surface is constructed without performing a painting step.

7. The method of claim 6, wherein the constructing comprises forming a starting material into a shape that when cooled and hardened is a black plastic solid or a dark plastic solid.

8. The method of claim 6, wherein the constructing comprises forming a plastic selected from the group consisting of a polyarylamide; a polyetherimide; a Polyether ether ketone (PEEK) and a liquid crystal polymer.

9. The method of claim 6, comprising formulating a plastic resin with or without glass.

10. The method of claim 6, comprising formulating the plastic resin without glass and thereby producing a plastic that is unfilled with glass.

11. The method of claim 10, comprising formulating the plastic resin with glass and producing a glass-filled plastic in range of 0.01 to 60% glass-filled.

12. The method of claim 6, comprising a step of pellet-melting.

13. The method of claim 12, wherein the pellet-melting step comprises melting natural-colored polymer pellets.

14. The method of claim 12, wherein the pellet-melting step comprises melting dark polymer pellets or black polymer pellets.

15. The method of claim 12, further comprising, while polymer pellets are in a melted state, adding at least one pigment and thereby darkening the melted polymer.

16. The method of claim 15, wherein the pigment-adding step comprises pigment addition until the melted polymer has been blackened.
